(19) United States
(12) Patent Application Publication
(10) Pub. No.: US 2021/0052775 A1
(43) Pub. Date: Feb. 25, 2021

(54) METHOD OF MANUFACTURING PARTICULATE TISSUE PRODUCTS

(71) Applicant: LifeCell Corporation, Madison, NJ (US)

(72) Inventors: Mark DeCaro, Millstone Township, NJ (US); Timothy Roock, Bordentown, NJ (US)

(21) Appl. No.: 16/997,465

(22) Filed: Aug. 19, 2020

Related U.S. Application Data
(60) Provisional application No. 62/889,343, filed on Aug. 20, 2019.

(51) Int. Cl. A61L 27/36

(52) U.S. Cl. CPC A61L 27/3683; A61L 2430/40; A61L 27/362; A61L 27/3633

(57) ABSTRACT

The present disclosure relates to a method for manufacturing particulate tissue products. The methods can include cutting a sheet of tissue matrix into elongated strips. In various embodiments, the method for manufacturing particulate tissue product further includes bundling the strips and slicing the bundle into particulate tissue product. The particulates may have improved properties, such as a uniform size distribution.

This application claims priority under 35 USC § 119 to U.S. Provisional Application No. 62/889,343, which was filed on Aug. 20, 2019 and is herein incorporated by referenced in its entirety.

The present disclosure relates to a tissue product, including particulate tissue products and methods of making such products.

Various tissue-derived products are used to regenerate tissue, facilitate wound healing, or otherwise treat diseased or damaged tissues and organs. For example, tissue matrices are tissue-derived products that may be used during surgery to fill voids, connect tissues, or support implanted materials.

Tissue matrices can include tissue grafts or decellularized tissues provided in a variety of forms. For example ALLODERM® and STRATTICE™ (Lifecell Corporation, Branchburg, N.J.) are tissue matrix products provided in flexible sheet configurations. Sheets of tissue matrices can be beneficial and provide lifesaving advantages. However, tissue matrix sheets are not ideal for some uses. For example, although valuable as tissue regenerative materials for load-bearing (e.g., hernia or breast support), such sheets may not be ideal for filling irregular voids.

Tissue matrices may also be provided in particulate forms, which can be used as tissue filler. These particulate tissue products are useful when filling small voids or for injection. For example, facial reconstruction or rejuvenation procedures can use particulate tissue products that are injected using small-gauge needles. Further, although existing particulate tissue products are useful for some applications, improved methods for generating the particulate forms may be desirable.

The present application provides methods for manufacturing particulate tissue products that can be used as tissue filler. The method comprises selecting a tissue matrix and cutting the tissue matrix into elongated tissue matrices. The method further comprises combining the one or more elongated tissue matrices into a group of elongated tissue matrices and cutting the group of elongated tissue matrices to produce a group of particulate tissue products.

### BRIEF DESCRIPTION OF THE DRAWINGS

The present invention is illustrated by way of example, and not limitation, in the accompanying figures wherein:

FIG. 1 illustrates a sheet of tissue matrix that may be used in conjunction with the devices and methods of the present disclosure.

FIG. 2 illustrates a cutting tool used to create an elongated strip of tissue matrix, according to various embodiments of the present disclosure.

FIG. 3 illustrates a sheet of tissue matrix and a cutting tool used to create an elongated strip of tissue matrix, according to various embodiments of the present disclosure.

FIG. 4 illustrates an elongated tissue matrix manufactured formed from a sheet of tissue matrix using a cutting tool, according to various embodiments of the present disclosure.

FIG. 5 illustrates an alternate configuration for a cutting tool used to create elongated strips of tissue matrix, according to various embodiments of the present disclosure.

FIG. 6 illustrates a bundle of elongated tissue matrices manufactured according to various embodiments of the present disclosure.

FIG. 7 illustrates a prepared sample including a bundle of elongated tissue matrices, and a slicing machine, according to various embodiments of the present disclosure.

FIG. 8 illustrates a graph of a particle size distribution chart two particle size distribution curves.

FIGS. 9A-9C illustrate magnified views of particulate tissue products provided in multiple thicknesses, prepared according to various embodiments of the present disclosure.

FIGS. 9D-9F illustrate suspensions comprising particulate tissue products of multiple thicknesses, prepared according to various embodiments of the present disclosure.

FIG. 10 illustrates an exemplary application for particulate tissue products prepared according to various embodiments of the present disclosure.

### DESCRIPTION OF CERTAIN EXEMPLARY EMBODIMENTS

Reference will now be made in detail to various embodiments of the disclosed devices and methods, examples of which are illustrated in the accompanying drawings. Wherever possible, the same reference numbers will be used through the drawings to refer to the same or like parts. The drawings are not necessarily to scale.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

In this application, the use of the singular includes the plural unless specifically stated otherwise. Also in this application, the use of “or” means “and/or” unless stated otherwise. Furthermore, the use of the term “including,” as well as other forms, such as “includes” and “included,” are not limiting. Any range described here will be understood to include the endpoints and all values between the endpoints.

The section headings used herein are for organizational purposes only and are not to be construed as limiting the subject matter described. All documents, or portions of documents, cited in this application, including, but not limited to patents, patent applications, articles, books, and treatises, are hereby expressly incorporated by reference in their entirety for any purpose. To the extent publications and patents or patent applications incorporated by reference contradict the invention contained in the specification, the specification will supersede any contradictory material.

The present disclosure relates generally to methods for producing particulate tissue products with desired shapes and sizes. The methods and devices are implemented to transform sheets of tissue matrix into elongated tissue matrices and transform the elongated tissue matrices into particulate tissue products. The methods allow formation of particulates with specific or narrow size distributions, and allow formation of precise sizes with minimal amounts of, or entirely without, undesirably small or large particles. Such improved control of size distribution can improve control of injection, flowability, or biologic response (e.g., by controlling degradation rate).

According to the methods provided herein, tissue matrices can be formed into elongated strips or noodle-like parts by cutting. The elongated strips or noodle-like parts can further be formed into particulate materials by cutting or slicing the strips or noodle-like parts.

Various methods of producing particulate tissues are known, but often such processes include grinding or milling. And although such processes are effective at producing particulate tissue fillers, such processes have some drawbacks. For example, grinding or milling can impart damage to the particles that results in less than optimal biologic response. Furthermore, using grinding or milling processes, the resultant materials may have a wide particle size distribution, which can create challenges in controlling injectate viscosity and may further result in undesired inflammation.

The presently disclosed manufacturing methods enable superior control of both particle size and size distribution. Furthermore, the disclosed methods enable control and optimization of various characteristics of particulate injectate, such as rheological behavior (e.g. viscosity), density, and injectability, and result in an improved biologic response caused from the uniformity of particle size. Additionally, particles made according to methods of the present disclosure comprise smooth exterior surfaces, whereas particles made by grinding or milling processes tend to have fibrous exterior surfaces. When stored in a syringe, over time such fibrous particles tend to aggregate, making them difficult to inject through a syringe needle. The particles of the present disclosure comprise smoother exteriors and have longer shelf lives because they are less susceptible to aggregation.

The tissue matrix materials used to produce the tissue products described herein can be derived from a variety of materials. For example, elongated tissue matrices can be formed from ALLODERM® or STRATTICE™ (Lifecell Corp., Branchburg, N.J.), which are human and porcine acellular dermal matrices, respectively. Furthermore, a number of tissue matrix materials are described by Badylak et al. These tissue matrix materials may be processed as described herein to produce particulate tissue products. Accordingly, Badylak et al., “Extracellular Matrix as a Biological Scaffold Material: Structure and Function,” Acta Biomaterialia (2008), doi:10.1016/j.actbio.2008.09.013, is hereby incorporated by reference in its entirety.

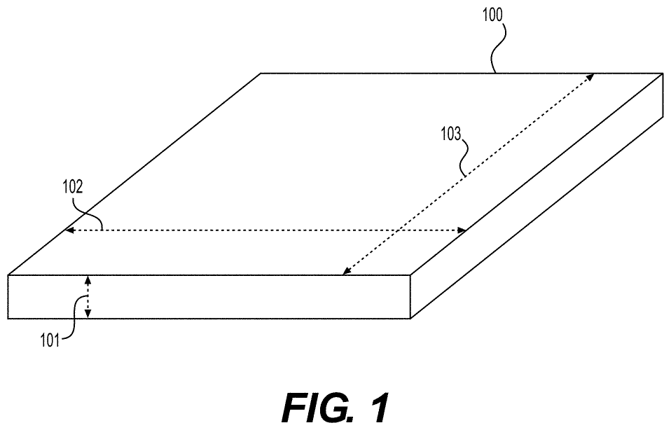

In certain embodiments, elongated tissue matrices can be formed from tissue matrices provided in sheet configurations. FIG. 1 illustrates a sheet of tissue matrix 100 that may be used in conjunction with the present devices and methods. The sheet of tissue matrix 100 comprises a thickness 101, length 102, and width 103. Further, although described particularly with respect to sheets, tissue matrices in other shapes or bulk forms may be used as the starting materials.

Elongated tissue matrices may be manufactured in a variety of ways. For example, sheets of tissue matrix can be sliced into elongated structures with a bladed instrument, such as a scalpel, knife, or other device incorporating a blade. Elongated tissue matrices may also be manufactured in a variety of configurations. For example, elongated tissue matrices can be provided with approximately circular, triangular, square, rectangular, higher-order polygonal, or amorphous cross-sections. Additionally, the cross-sections may be approximately constant or may vary over the length of the elongated tissue matrix.

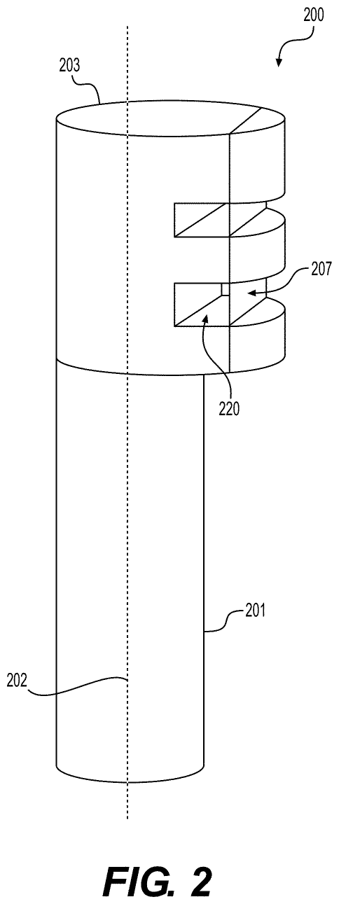

In certain embodiments, elongated tissue matrices may be manufactured using specially constructed tools. For example, FIG. 2 illustrates an exemplary cutting tool 200. Cutting tool 200 comprises a handle 201, longitudinal axis 202, and tool head 203. Tool head 203 comprises at least one aperture 220 and blade 207. As illustrated in FIG. 2, the at least one aperture 220 lies on the periphery of cutting tool head 203, and blade 207 comprises one surface of aperture 220.

Handle 201 of cutting tool 200 may be provided in a variety of shapes and configurations. Additionally, cutting tool head 203 comprises at least one aperture 220, which may be provided in varying forms and quantities. For example, tool head 203 may comprise apertures 220 of different sizes to enable tissue processing of sheets of tissue matrix 100 with varying thicknesses 101. Additionally, the aperture 220 may assume a variety of shapes. For example, aperture 220 may assume the shape of a semi-cylinder, rectangular, square or triangular structure, or various other forms. The size and shape of aperture 220 may be selected to determine the cross-section of the elongated tissue matrix, and in turn, the small particulate tissue products produced therefrom.

According to various embodiments, the elongated tissue matrices may be provided with various cross sections. As recited above, the cross-sections may include circular, triangular, square, rectangular, higher-order polygonal, or generally amorphous configurations. In some embodiments, cylindrical elongated tissue matrices possessing circular cross-sections, when manufactured into particulate tissue matrices will have a substantially disk-like shape. In various other embodiments, the elongated tissue matrices may be provided with square cross sections, and, when manufactured into particulate form, these tissue products will have sheet-like shapes. Accordingly, the size and shape of the particulate tissue products disclosed herein will be determined, in part, by aperture 220 of cutting tool 200.

Blade 207 may be connected to tool head 203 using a variety of mechanical or chemical fixing means. In one embodiment, blade 207 of cutting tool 200 may be secured to the cutting tool so that the blade 207 may be attached and detached from cutting tool 200 one or multiple times. Additionally, blade 207 may be provided in a variety of configurations. For example, multiple blades 207 may be aligned in a rake-like pattern such that a single pass of the cutting tool 200 along the length 102 or width 103 of the sheet of tissue matrix 100 can produce multiple elongated tissue matrices.

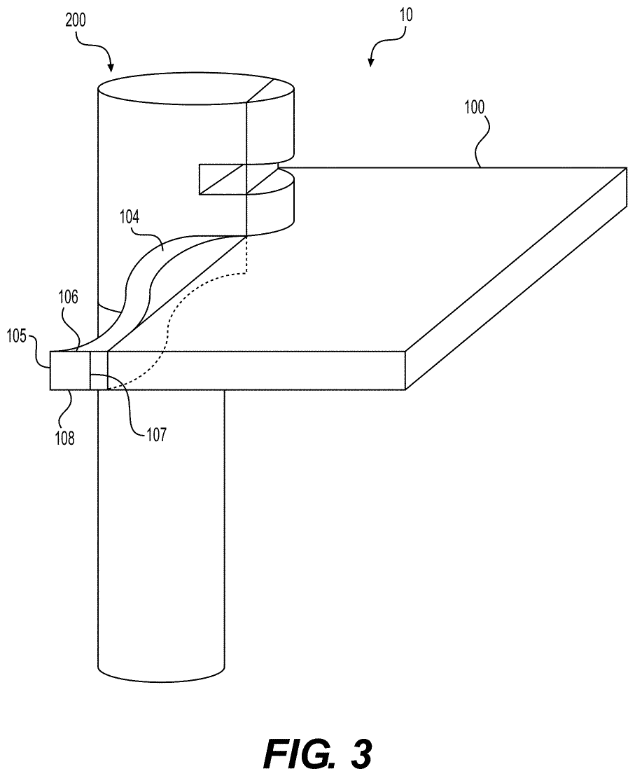

FIG. 3 illustrates a system 10 comprising a sheet of tissue matrix 100 and a cutting tool 200, used to manufacture elongated tissue matrices, according to various embodiments of the present disclosure. The method disclosed herein comprises advancing a portion 104 of the sheet of tissue matrix 100 through an aperture 220 of cutting tool 200 to form a continuous, elongated strip of tissue matrix.

In some embodiments, the method of manufacturing elongated tissue matrices comprises advancing the cutting tool 200 along the length 102 and width 103 of the sheet of tissue matrix 100, positioning the sheet of tissue matrix 100 such that the thickness 101 of the sheet of tissue matrix 100 through aperture 220, wherein the thickness 101 does not exceed the height of the aperture 220. Sheet of tissue matrix 100 and cutting tool 200 may be manipulated in a variety of ways to produce the desired size, shape, and length of elongated tissue matrix.

In certain embodiments of the present disclosure, to begin executing the method disclosed herein, a pre-cut portion 104 of the sheet of tissue matrix 100 is fed through aperture 220 of cutting tool 200. The sheet of tissue matrix 100 is oriented such that the thickness 101 of the sheet 100 is substantially parallel to the blade 207, which contacts with the sheet of tissue matrix 100. For example, the sheet of tissue matrix 100 and the cutting tool 200 may be positioned such that edges 105, 106, and 108 of the portion 104 of the sheet of tissue matrix 100 align with the inner surface of aperture 220.

In certain embodiments, the method of manufacturing elongated tissue matrices comprises applying tension to the portion 104 of the sheet of tissue matrix 100 exiting the aperture 220 to continue advancing more portions of the sheet of tissue matrix 100 through the aperture 220. Tension is applied until a sufficient length of elongated tissue matrix is produced. Tension may be applied to the portion 104 of the sheet of tissue matrix 100 exiting aperture 220 in a variety of ways. For example, cutting tool 200 may be mounted to a stand and the portion 104 of the sheet of tissue matrix 100 exiting aperture 220, may be grasped and placed under tension using any suitable gripping device. A suitable gripping device may include tweezers, forceps, pliers, or the like. Additionally, tension forces may be generated using an automated process.

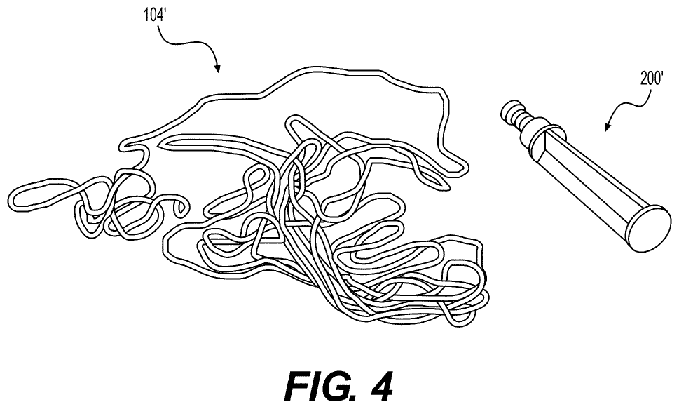

In an exemplary embodiment of the present disclosure, FIG. 4 illustrates an elongated tissue matrix 104′ manufactured from a sheet of tissue matrix. Portions of a sheet of tissue matrix were advanced through the aperture of cutting tool 200′ until a desired number of elongate tissue matrices were produced. Resultant elongated tissue matrix 104′ has a substantially similar cross section along its length.

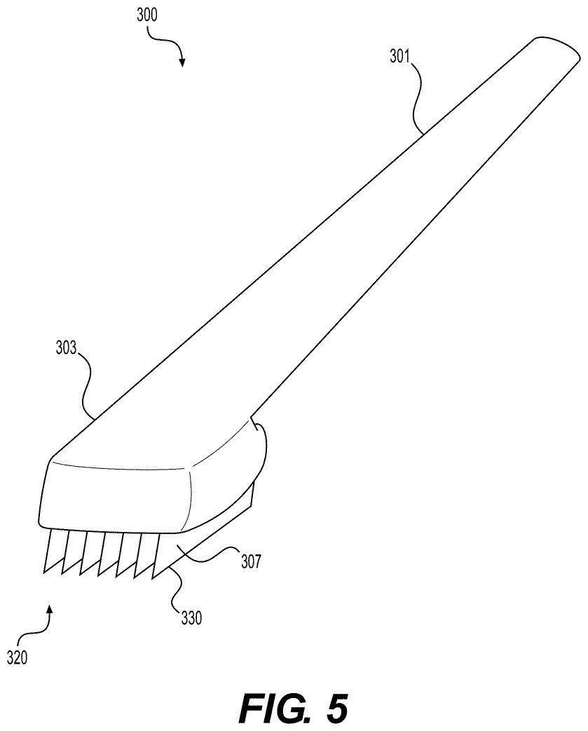

In certain embodiments alternate configurations for a cutting tool used to create elongated strips of tissue matrix are provided. FIG. 5 illustrates cutting tool 300, which in some configurations, may comprise handle 301 and tool head 303. Tool head 303 may comprise multiple apertures 320 and blades 307. Blades 307 may be positioned with respect to tool head 303 in accordance with clinical need. For example, if 3 mm wide strips are desired, blades 307 may be positioned at 3 mm intervals (or slightly larger to account for tissue lost due to blade thickness). Blades 307 may be removably attached to tool head 303 to enable replacement of dulled or damaged blades.

Blades 307 may be attached to tool head 303 at one or more locations. For example, in various configurations, blades 307 may be attached to tool head 303 only at one edge of blade 307. In this configuration, apertures 320 may comprise three sides, and have one open side. Thus, cutting tool 300 may be used with a sheet of tissue matrix positioned on a flat surface. Cutting tool 300 may be pressed into tissue matrix 100 to cut into tissue matrix 100. Afterward, cutting tool 300 may be pulled or dragged through tissue matrix 100 to produce elongated strips of tissue matrix. To maximize yields, blades 307 may be configured to cut tissue matrix 100 without causing undue damage to tissue matrix 100.

In various embodiments, apertures 320 may traverse tool head 303 in an orientation substantially parallel with the length of handle 301. Tool head 303 may comprise multiple blades at varying intervals. In various embodiments, blades 307 may be adjustable within tool head 303 so that the spacing between them may be changed to suit clinical need. For example, an operator may use fewer blades spaced at larger interval distances to achieve wider elongated tissue matrices. Alternatively, an operator may add multiple blades at small interval lengths to produced narrow elongated matrices. In various embodiments, cutting edges 330 of blades 307 comprise one, two, or three edges of blades 307. Multiple cutting edges 330 of blades 307 provide greater cutting capabilities when using cutting tool 300.

In certain embodiments, the method of manufacturing elongated tissue matrices may further comprise treating elongated tissue matrix 104, 104′ to alter the physical or chemical properties thereof. For example, the tissue matrix may be cross-linked with compounds to increase the density and mechanical properties of the elongated tissue matrices 104, 104′. Also, the tissue matrix may be treated with additional agents. These agents may comprise an anti-inflammatory agent, an analgesic, or any other biocompatible, therapeutic agent. In certain embodiments, the additional agent can comprise at least one added growth or signaling factor (e.g., a cell growth factor, an angiogenic factor, a differentiation factor, a cytokine, a hormone, and/or a chemokine). These additional agents can promote native tissue migration, proliferation, and/or vascularization, to increase the likelihood of implantation success.

After production of the elongated tissue matrices, the matrices can be further processed to produce particulates. The elongated tissue matrices can be assembled into a bundle. In some cases, the bundle can be rigidified, and then sliced to form particulates.

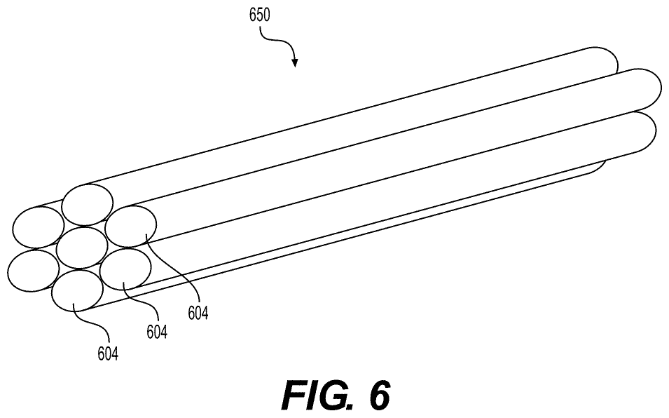

As discussed previously, elongated tissue matrices manufactured according to the disclosed methods contain substantially similar cross-sectional dimensions along their length. Accordingly, elongated tissue matrices may be cut and assembled into bundles. FIG. 6 illustrates a bundle 650 of elongated tissue matrices 604. Although bundle 650 is depicted in FIG. 6 as comprising seven elongated tissue matrices 604, according to various embodiments of the present disclosure, bundle 650 can include multiple elongated tissue matrices 604. Bundle 650 can include, but is not limited to, 2, 3, 4, 5, 10, 15, 20, 25, 30, or 50 (or more) elongated tissue matrices 604. In various embodiments, the size of bundle 650 will be governed by the size of the slicing machine used to cut bundle 650.

In certain embodiments, bundle 650 may comprise elongated tissue matrices with the same cross-sectional dimensions. In other embodiments, bundle 650 may comprise elongated tissue matrices with two or more distinct cross-sectional dimensions. For example, in certain embodiments, elongate tissue matrices 604 with two distinct cross-sections can be combined in the same bundle 650. After bundle 650 is processed into particulate tissue product using methods of the present disclosure, the resultant particulate tissue product will comprise two precise size distributions. A particulate tissue product with particles of two distinct sizes may enhance spreadability of the particulate tissue product in vivo. Alternatively, more than two sizes can be used. Furthermore, particulates of differing sizes can be produced separately and then mixed to produce a desired mixture of sizes.

According to various embodiments, the slicing machine used with the disclosed methods can include various devices capable of producing thin slices of material. For example, the slicing machine can include rotating fan blade cutters, deli slicers, mandolins, or microtomes in various configurations and embodiments.

In one embodiment, the slicing machine comprises a cryostat microtome. For use with this device, bundle 650 is frozen to provide rigidity to the elongated tissue matrices 604. The cryostat microtome includes a cooling chamber capable of maintaining low temperatures, sufficient to keep the bundle 650 frozen while the microtome is in use. Accordingly, while the tissue is subjected to shear forces generated by the microtome blade, the cross section of each frozen elongated tissue matrix 604 remains unchanged.

In another embodiment, the slicing machine comprises a standard microtome, which can be used when portions of the bundle 650 are embedded in paraffin wax, embedding compounds, such as optimal cutting temperature (“OTC”) compound, or otherwise stabilized to allow cutting. Embedding the flexible tissue in paraffin or embedding compounds provides the elongated tissue matrices 604 and bundle 650 with sufficient rigidity to withstand microtome slicing without resulting in changes to its cross section.

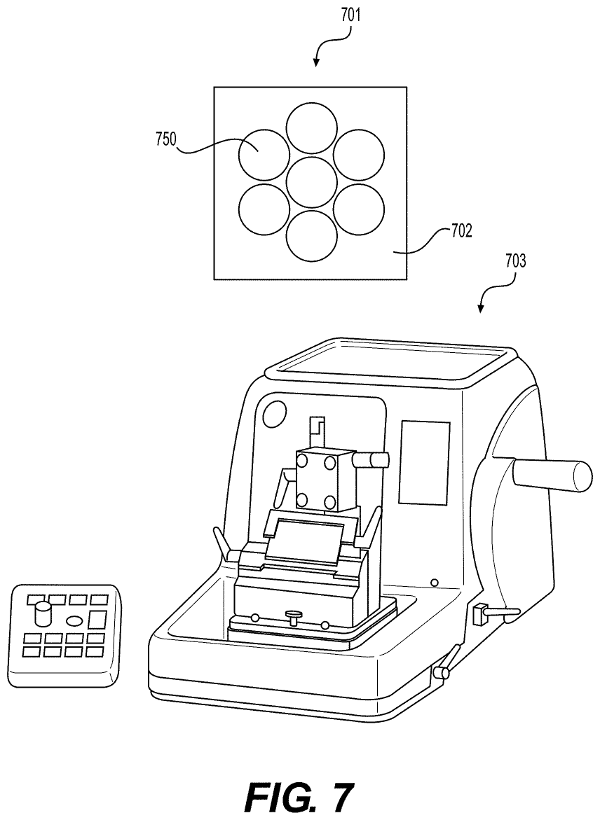

FIG. 7 illustrates a prepared microtome sample 701 including a bundle of elongated tissue matrices 750 embedded in paraffin wax 702, according to various embodiments of the present disclosure. Sample 701 may be used with microtome 703 according to methods known in the art. The settings of microtome 703 can be adjusted to change the thickness of the particulate tissue products produced therefrom. Particulate tissue product thicknesses can include, but are not limited to 2, 3, 4, 5, 10, 15, 20, 25, 30, 35, 40, 45, 50, 60, 70, 80, 90, 100, 150, 200, or more μm. After slicing, particulate tissue products can be further processed by submerging the particulates into a washing solution.

In some embodiments, the particulate tissue product of the present disclosure comprises a substantially uniform particulate size and shape. Particle size distribution can be visualized using a curve where the chart's x-axis displays sizes in length and the y-axis displays volume percentage. As used herein, particulate tissue product comprising a “substantially uniform particulate size and shape” is a particulate tissue product wherein the particle size distribution presents as a narrow peak, higher than it is wide.

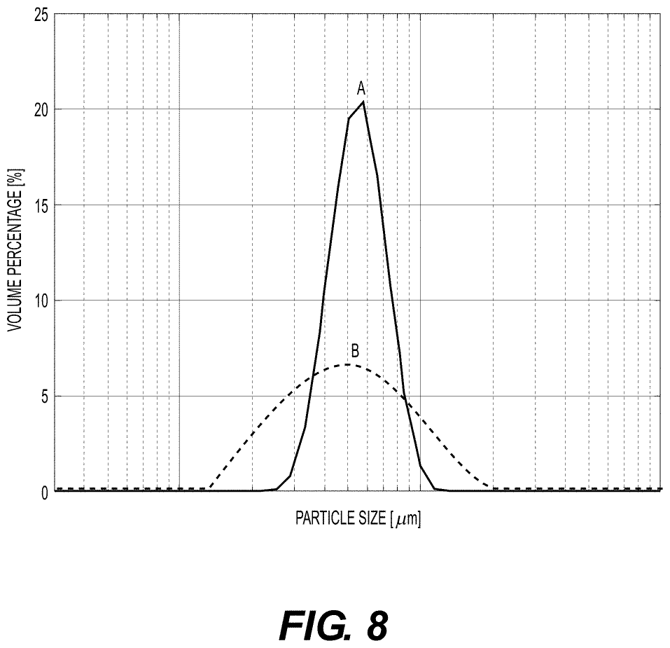

For example, FIG. 8 illustrates a particle size distribution chart with two particle size distribution curves. Curve A represents a narrow size distribution wherein the majority of the particles are the same or very similar size. Curve A represents the particle size distribution for a substantially uniform particulate tissue product. Curve B represents a wide size distribution where the particles include a large range of sizes. An advantage of the present disclosure over existing particulate tissue manufacturing systems is that the particle size distribution of the particulate tissue product of the present disclosure has a particle size distribution similar to that of Curve A of FIG. 8.

### Examples

To study the effects of particulate tissue product size and thickness in clinical applications, elongated tissue matrices 604 prepared according to methods of the present disclosure were sliced using a microtome. Elongated tissue matrices 604 were prepared from a sheet of tissue matrix 100 that had a thickness of 1 mm (1000 μm). The cutting tool 200 used in the exemplary embodiment had an aperture width of 0.5 mm (500 μm). Thus, the resultant height and width of elongated tissue matrix 604 produced in the exemplary embodiment were 1 mm and 0.5 mm, respectively.

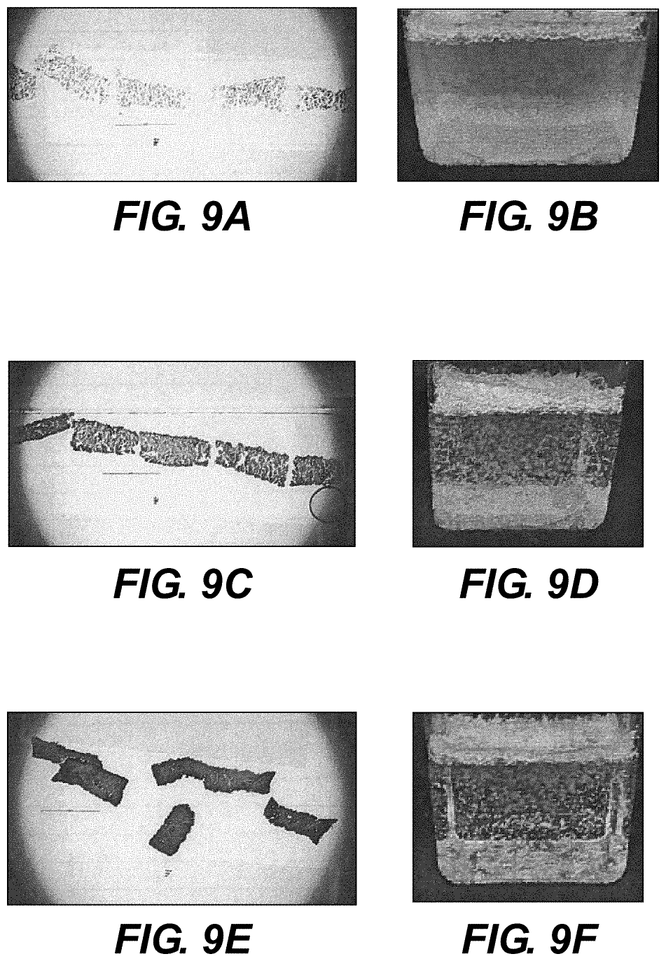

Elongated tissue matrices 604 were prepared for the microtome, according to various embodiments of the present disclosure and cut into multiple thicknesses. The stranded samples measured approximately 1 mm×0.5 mm in cross section, and were sliced in various thicknesses. FIGS. 9A-9C illustrate magnified views of particulate tissue products in three thicknesses. FIG. 9A illustrates a magnified view of particulate tissue product 10 μm in thickness. FIG. 9B illustrates a magnified view of particulate tissue product 50 μm in thickness. FIG. 9C illustrates a magnified view of particulate tissue product 100 μm in thickness. As can be observed, the thinner the particulate tissue product, the more light is allowed to pass through the sample. Notably, although the particulate tissue products illustrated in FIGS. 9A-9C vary in thickness, the height and width of these products remains substantially similar. Accordingly, the FIGS. 9A-9C illustrate that the methods of the present disclosure result in particulate tissue product with substantially similar sizes.

The ability to manufacture particulate tissue product with narrow size distributions provides numerous clinical advantages. In one instance, because the present disclosure provides methods for controlling both the shape and size of the cross section of the elongated tissue matrices, and the thickness of the particulate tissue product produced therefrom, surgeons can optimize particulate tissue products to specific clinical applications. For example, when particulate tissue products are used to fill deep wrinkles and large voids, large-size tissue particulates can be used. When contouring fine lines and small voids, small-size tissue particulates may provide better clinical results.

In further example, characteristics such as tissue regeneration, vascularization, immune response, and native tissue ingrowth, can be optimized by changing tissue particle size with the disclosed methods. For example, ground tissue matrix, while providing clinical benefits, often results in particulate tissue product with a large particle size distribution. As a result, some tissue particles are small enough to be digested by leukocytes, causing an enhanced immune response. Manufacturing the particulate tissue product such that each particle is too large for leukocytes to digest, could improve the immune response of the injectate.

In another example, controlling the size of particulate tissue product would result a less viscous material capable of passing through small gauge needles. Cosmetic or contouring procedures in the face and neck involve small injections of particulate tissue product into the face or neck or a patient to correct, enhance, or reconstruct facial features. Common procedures may include, for example, lip augmentation procedures or the treatment of facial rhytids, such as nasolabial folds, mesolabial folds, oral commissures, periorbital lines, and glabellar lines. Since patients undergoing minimally invasive cosmetic procedures are not typically sedated, small needles are desirable to minimize patient anxiety, pain, and scarring. Thus, particulate tissue product made according to the presently disclosed methods can be manufactured to pass through small gauge needles, for example, 24, 25, 26, 27, 28, 29, 30, 31, and 32 gauge needles.

To produce material suitable for injection, particulate tissue product, such as those illustrated in FIGS. 9A-9C, can be suspended in solution. For example, FIGS. 9D-9F illustrate suspensions comprising particulate tissue products of similar height and width, but of varying thicknesses. As discussed above, the thicknesses of the particulate tissue products are controlled by the microtome used. 9D illustrates a suspension comprising 10 μm thick particulate tissue product. 9E illustrates a suspension comprising 50 μm thick particulate tissue product. 9F illustrates a suspension comprising 100 μm thick particulate tissue product.

To determine the smallest gauge needle that could be used with the illustrated suspensions, small volumes of each suspension were inserted into the barrel of a 1 ml syringe. Multiple needle sizes were attached to the syringe to determine if the particulate tissue product suspensions could pass therethrough. The material containing 10 μm thick particulate tissue product, illustrated in FIG. 9D, successfully passed through a 27 gauge needle. However, the same material was not able to pass through a 30 gauge needle.

Next, additional 10 μm particles as illustrated in FIG. 9A, were washed, centrifuged, and mixed with Hyaluronic Acid to make 25% and 12.5% solid content suspensions. The 25% solid content suspension comprising 10 μm particles successfully passed through a 27 gauge needle. The 12.5% solid content suspension comprising 10 μm particles successfully passed through both 27 gauge and 30 gauge needles. These results indicate that 10 μm tissue product particles, provided in 12.5% solid suspensions can be used in cosmetic procedures of the face and neck, because they can pass through sufficiently small gauge needles.

According to certain embodiments, the particulate tissue product can be prepared for clinical use. For example, the particulate tissue product can be sterilized and packaged in vials or syringes to be brought into the commercial market. In various embodiments, the particulate tissue product can be provided in numerous forms, including slurries and suspensions with multiple solid contents. The particulate tissue products can be tailored for use in various procedures or with various needle gauges so that the surgeons may customize use thereof. For example, 25% solid content, 20 μm particulate tissue product can be well suited to contour rhytids of the neck, whereas 12.5% solid content, 8 μm particulate tissue product can be well suited to contour fine rhytids present in thin eye skin, such as crow's feet.

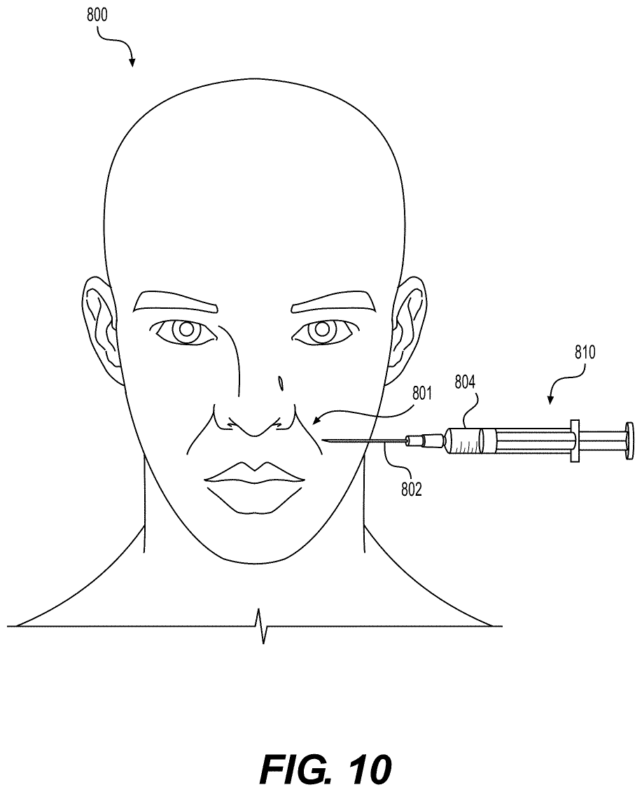

According to certain embodiments, a diagram of one such procedure is illustrated in FIG. 10, which depicts a cosmetic procedure to contour the nasolabial folds 801 of patient 800. After patient preparation, a surgeon may add particulate tissue product suspension 804 into syringe 810. The surgeon may then attach a small, 30 gauge needle to the barrel of syringe 810. With minimum pain and scarring to the patient, the surgeon may inject particulate tissue matrix 804 into nasolabial fold 801 of patient 800. For example, the syringe needle can pierce the skin of the patient at an injection site and a syringe plunger can be depressed into the body of the syringe to expel particulate tissue product into the injection site.

A benefit of injecting particulate tissue product manufactured according the methods of the present disclosure has been observed in shallow injections. For example, shallow injections of particulate tissue product manufactured according to methods of the present disclosure were administered to porcine skin. The injection site was examined post-injection and the injected tissue blended smoothly with the host tissue.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of manufacturing particulate tissue products comprising:
selecting a tissue matrix;
cutting a tissue matrix into elongated tissue matrices;
combining the one or more elongated tissue matrices into a group of elongated tissue matrices; and
cutting the group of elongated tissue matrices to produce a group of particulate tissue products.

2. The method of claim 1, wherein the elongated tissue matrices each have an approximately uniform cross sectional area.

3. The method of claim 1, further comprising treating one or more elongated tissue matrices to alter the physical or chemical properties of the elongated tissue matrices.

4. The method of claim 1, wherein the group of elongated tissue matrices comprises a bundle of elongated tissue matrices.

5. The method of claim 1, wherein the group of elongated tissue matrices is rigidified.

6. The method of claim 5, wherein the group of elongated tissue matrices is rigidified by fixing the group of elongated tissue matrices in embedding medium.

7. The method of claim 1, wherein cutting the group of elongated tissue matrices to produce particulate tissue products comprises cutting along a plane approximately perpendicular to the a axis of the group of elongated tissue matrices

8. The method of claim 1, wherein a microtome is used to cut the group of elongated tissue matrices.

9. The method of claim 8, wherein the microtome used to cut the group of elongated tissue matrices comprises a cryostat microtome.

10. The method of claim 1, wherein the group of particulate tissue products has a narrow size distribution.

11. The method of claim 1, wherein the particulate tissue products are suspended in a solution.

12. The method of claim 1, wherein the tissue matrix comprises the partially or fully decellularized tissue matrix from at least one of bone, skin, dermis, intestine, vascular, urinary bladder, tendon, ligament, muscle, fascia, neurologic tissue, vessel, liver, heart, lung, kidney, or cartilage tissue.

13. The method of claim 1, wherein the tissue matrix comprises at least one dermal acellular tissue matrix.

14. The method of claim 1, wherein the tissue matrix lacks substantially all alpha-galactose moieties.

15. The method of claim 1, further comprising one or more viable and histocompatible cells.
